(19) United States
(12) Patent Application Publication
(10) Pub. No.: US 2020/0376262 A1
(43) Pub. Date: Dec. 3, 2020

(54) SYSTEMS AND METHODS FOR MAKING AND USING IMPLANTABLE ELECTRICAL/OPTICAL STIMULATION LEADS AND SYSTEMS

(71) Applicant: Boston Scientific Neuromodulation Corporation, Valencia, CA (US)

(72) Inventors: Bryan Allen Clark, Forest Lake, MN (US); Elizabeth M. Annoni, White Bear Lake, MN (US)

(21) Appl. No.: 16/883,404

(22) Filed: May 26, 2020

Related U.S. Application Data
(60) Provisional application No. 62/854,506, filed on May 30, 2019.

(51) Int. Cl. A61N 1/05; A61N 5/06

(52) U.S. Cl. CPC A61N 1/0553; A61N 2005/063; A61N 5/0601; A61N 1/0556

(57) ABSTRACT

A stimulation lead includes light emitters disposed along the distal portion of the lead and stimulation electrodes disposed along the distal portion of the lead with a portion of the lead upon which at least one of the light emitters and at least one of the electrodes is disposed is arranged to form a coil or spiral. Another stimulation lead includes a lead body; a paddle or cuff attached to the lead body; light emitters disposed on the paddle or cuff; and stimulation electrodes disposed on the paddle or cuff. Yet another stimulation lead includes a lead body; a paddle or cuff attached to the lead body; at least one long electrode disposed on the paddle or cuff and extending at least 50% of a width or length of the paddle or cuff; and segmented stimulation electrodes disposed in at least one row or column on the paddle or cuff.

### CROSS-REFERENCE TO RELATED APPLICATIONS

This application claims the benefit under 35 U.S.C. § 119(e) of U.S. Provisional Patent Application Ser. No. 62/854,506, filed May 30, 2019, which is incorporated herein by reference.

### FIELD

The present disclosure is directed to the area of electrical and optical stimulation systems and methods of making and using the systems. The present invention is also directed to implantable electrical and optical stimulation leads for use with peripheral nerve stimulation and sensing, as well as methods of making and using the leads and stimulation systems.

### BACKGROUND

Electrical or optical stimulation systems can provide therapeutic benefits in a variety of diseases and disorders. For example, optical stimulation can be applied to the brain either externally or using an implanted stimulation lead to provide, for example, deep brain stimulation, to treat a variety of diseases or disorders. Optical stimulation may also be combined with electrical stimulation.

Stimulators have been developed to provide therapy for a variety of treatments. A stimulator can include a control module (for generating light or electrical signals sent to light sources in a lead), one or more leads, and one or more light sources coupled to, or disposed within, each lead. The lead is positioned near the nerves, muscles, or other tissue to be stimulated.

### BRIEF SUMMARY

One aspect is a stimulation lead that includes a lead body having a distal portion and a proximal portion; light emitters disposed along the distal portion of the lead body and configured to emit light; and stimulation electrodes disposed along the distal portion of the lead body; wherein a portion of the lead body upon which at least one of the light emitters and at least one of the electrodes is disposed is arranged to form a coil or spiral.

In at least some aspects, a plurality of the stimulation electrodes are segmented electrodes. In at least some aspects, all of the segmented electrodes are positioned on a surface of the lead which forms an interior surface of the coil or spiral. In at least some aspects, all of the segmented electrodes are positioned on a surface of the lead which forms an exterior surface of the coil or spiral.

Another aspect is a kit that includes any of the stimulation leads described above and a straightening stylet configured and arranged to straighten the coil or spiral of the stimulation lead when the straightening stylet is disposed in the stimulation lead.

A further aspect is a kit that includes any of the stimulation leads described above and a needle or cannula configured to receive the stimulation lead and to straighten the coil or spiral of the stimulation lead when the stimulation lead is received in the needle or cannula.

Yet another aspect is a stimulation lead that includes a lead body; a paddle or cuff attached to the lead body; light emitters disposed on the paddle or cuff and configured to emit light; and stimulation electrodes disposed on the paddle or cuff.

In at least some aspects, the light emitters are arranged in at least one row or column on the paddle or cuff and the stimulation electrodes are arranged in at least one row or column on the paddle or cuff. In at least some aspects, the light emitters are optical fibers, wherein each optical fiber is angled at a distal end of the optical fiber to emit light in a selected direction. In at least some aspects, the stimulation lead further includes at least one mechanical anchor coupled to each of the optical fibers and disposed in the paddle or cuff to hinder rotation of the optical fiber within the paddle or cuff.

In at least some aspects, the paddle or cuff is the cuff and the cuff is configured to apply pressure on a portion of a nerve disposed within the cuff to flatten the portion of the nerve. In at least some aspects, the paddle or cuff includes a body and one or more needle-like extensions extending from the body, wherein at least one of the stimulation electrodes is disposed on at least one of the needle-like extensions. In at least some aspects, includes a body and one or more needle-like extensions extending from the body, wherein at least one of the light emitters is configured to emit light from at least one of the needle-like extensions.

Another aspect is a system that includes any of the stimulation leads described above and a control module coupleable to the stimulation lead, the control module configured to direct the emission of the light from the light emitters of the lead and the delivery of electrical stimulation using the electrodes of the lead.

A further aspect is a stimulation lead that includes a lead body; a paddle or cuff attached to the lead body; at least one long electrode disposed on the paddle or cuff and extending at least 50% of a width or length of the paddle or cuff; and segmented stimulation electrodes disposed in at least one row or column on the paddle or cuff.

In at least some aspects, the at least one long electrode is two long electrodes and the segmented stimulation electrodes are disposed between the two long electrodes. In at least some aspects, the paddle or cuff is the cuff and the at least one long electrode extends at least 80% of the width or length of the cuff. In at least some aspects, the stimulation lead further includes at least one light emitter disposed on the paddle or cuff.

Yet another aspect is a system that includes any of the stimulation leads described above and a control module coupleable to the stimulation lead, the control module configured to direct the delivery of electrical stimulation using the electrodes of the lead.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of one embodiment of an electrical/optical stimulation system that includes a lead coupled to a control module, according to the invention;

FIG. 2A is a schematic side view of one embodiment of the control module of FIG. 1 configured and arranged to couple to an elongated device, according to the invention;

FIG. 2B is a schematic side view of one embodiment of a lead extension configured and arranged to couple the elongated device of FIG. 2A to the control module of FIG. 1, according to the invention;

FIG. 3 is a schematic overview of one embodiment of components of a stimulation system, including an electronic subassembly disposed within a control module, according to the invention;

FIG. 4A is a schematic side view of the distal portion of one embodiment of a straight electrical/optical stimulation lead;

FIG. 4B is a schematic side view of the distal portion of one embodiment of an electrical/optical stimulation lead with a coiled or spiral section;

FIG. 4C is a schematic side view of the distal portion of a second embodiment of an electrical/optical stimulation lead with a coiled or spiral section;

FIG. 4D is a schematic side view of the distal portion of a third embodiment of an electrical/optical stimulation lead with a coiled or spiral section;

FIG. 4E is a schematic side view of the distal portion of a fourth embodiment of an electrical/optical stimulation lead with a coiled or spiral section;

FIG. 5A is a schematic side view of the distal portion of one embodiment of a paddle lead for electrical/optical stimulation;

FIG. 5B is a schematic side view of the distal portion of one embodiment of a cuff lead for electrical/optical stimulation;

FIG. 5C is a cross-sectional view of a portion of the cuff lead of FIG. 5B;

FIG. 6A is a schematic side view of the distal portion of another embodiment of a cuff lead for electrical/optical stimulation;

FIG. 6B is a schematic side view of the distal portion of the cuff lead of FIG. 6A after flattening a portion of the nerve;

FIG. 7 is a schematic side view of the distal portion of another embodiment of an electrical/optical stimulation lead with needle-like extensions for stimulation; and

FIG. 8 is a schematic side view of the distal portion of one embodiment of a paddle lead for electrical stimulation.

### DETAILED DESCRIPTION

The present disclosure is directed to the area of electrical and optical stimulation systems and methods of making and using the systems. The present invention is also directed to implantable electrical and optical stimulation leads for use with peripheral nerve stimulation, as well as methods of making and using the leads and stimulation systems.

The systems described herein can be electrical stimulation systems, optical stimulation systems, or systems that can provide both electrical and optical stimulation. As described herein stimulation systems typically include a least one lead with one or more electrodes or light emitters (or any combination thereof) disposed along a distal portion of the lead and one or more terminals disposed along the one or more proximal portions of the lead. The leads can be, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 6,175,710; 6,224,450; 6,271,094; 6,295,944; 6,364,278; and 6,391,985; U.S. Patent Applications Publication Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; and 2013/0105071; and U.S. patent application Ser. Nos. 12/177,823 and 13/750,725, all of which are incorporated by reference in their entireties. Examples of optical stimulation systems with leads are found in, for example, U.S. Pat. No. 9,415,154 and U.S. Patent Application Publications Nos. 2013/0317573; 2017/0225007; 2017/0259078; 2018/0110971; 2018/0369606; and 2018/0369608, all of which are incorporated by reference in its entirety.

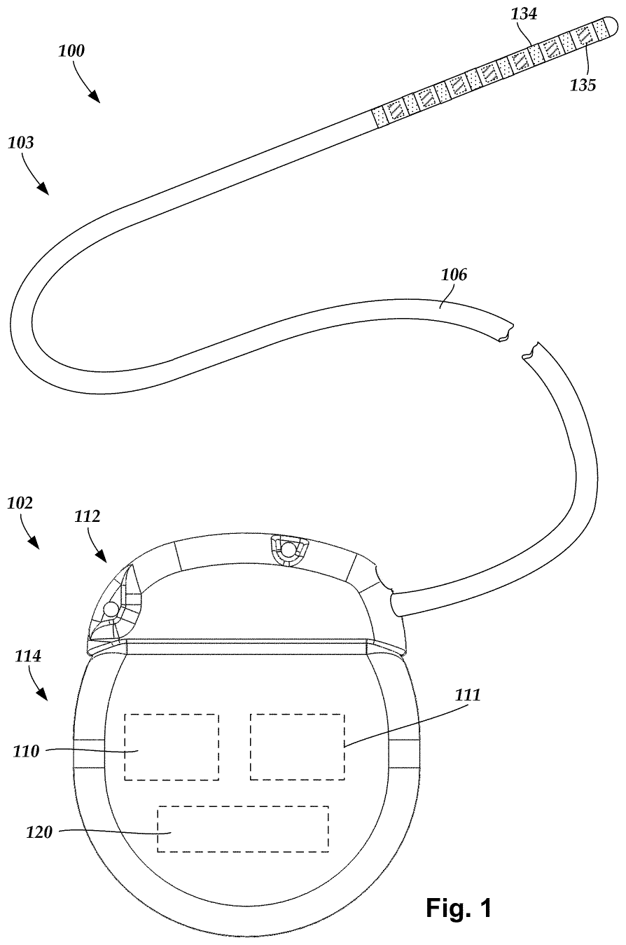

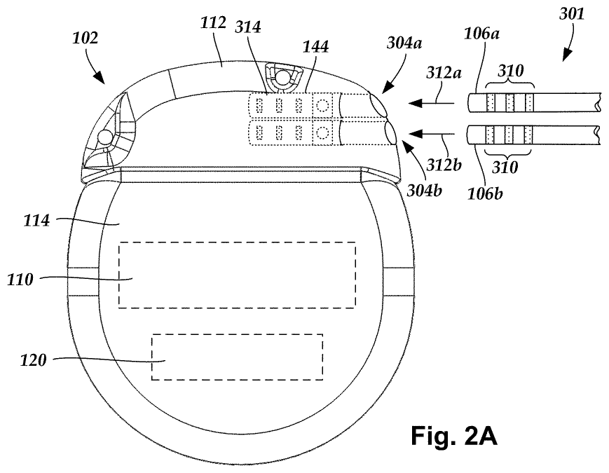

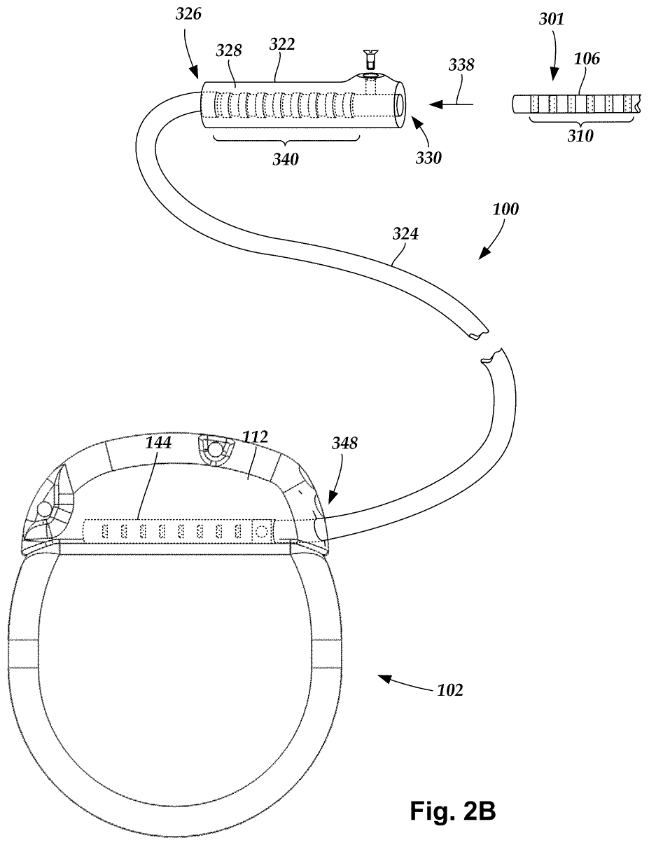

FIG. 1 illustrates schematically one embodiment of an electrical/optical stimulation system 100. The electrical/optical stimulation system includes a control module (e.g., a stimulator) 102 and a lead 103 coupleable to the control module 102 (such as an implantable or external pulse generator (IPG) or external trial stimulation (ETS)). The lead 103 includes one or more lead bodies 106. In FIG. 1, the lead 103 is shown having a single lead body 106. In FIG. 2B, the lead 103 includes two lead bodies. It will be understood that the lead 103 can include any suitable number of lead bodies including, for example, one, two, three, four, five, six, seven, eight or more lead bodies 106.

At least one light emitter 135 is provided along a distal portion of the lead 103. The light emitter 135 can be a light source, such as a light-emitting diode (“LED”), laser diode, organic light-emitting diode (“OLED”), or the like, or can be a terminus of a light transmission element, such as an optical fiber, in which case the light source is distant from the distal portion of the lead (for example, in the control module or in a proximal portion of the lead). Any suitable number of light emitters 135 can be disposed on the lead including, for example, one, two, three, four, five, six, seven, eight, nine, ten, eleven, twelve, fourteen, sixteen, twenty-four, thirty-two, or more light emitters 135.

The lead also includes electrodes 134 disposed along the lead body 106, and one or more terminals (e.g., 310 in FIG. 2A-2B) disposed along each of the one or more lead bodies 106 and coupled to the electrodes 134 by conductors (not shown). In at least some embodiments, one or more terminals (e.g., 310 in FIG. 2A-2B) may also be used to convey electrical signals to a light source that acts as the light emitter 135 by conductors (not shown) extending along the lead. In at least some embodiments, the system and electrodes may also be arranged for sensing action potentials, tissue properties, or other signals using the electrodes. As an example, in at least some embodiments, the system may sense actions potentials or other electrical signals or properties using the electrodes while delivering optical simulation using the light emitters.

The electrodes 134 and terminals (e.g., 310 in FIG. 2A-2B) can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium. Any suitable number of electrodes 134 and terminals (e.g., 310 in FIG. 2A-2B) can be disposed on the lead including, for example, one, two, three, four, five, six, seven, eight, nine, ten, eleven, twelve, fourteen, sixteen, twenty-four, thirty-two, or more electrodes 134 and terminals (e.g., 310 in FIG. 2A-2B).

The lead 103 can be coupled to the control module 102 in any suitable manner including directly or indirectly attached or coupled wirelessly. In some embodiments, the lead is permanently attached to the control module 102. In other embodiments, the lead can be coupled to the control module 102 by a connector (e.g., connector 144 of FIG. 2A). In FIG. 2A, the lead 103 is shown coupling directly to the control module 102 through the connector 144. In at least some other embodiments, the lead 103 couples to the control module 102 via one or more intermediate devices, as illustrated in FIG. 2B. For example, in at least some embodiments one or more lead extensions 324 (see e.g., FIG. 2B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the stimulation system 100 includes multiple elongated devices disposed between the lead 103 and the control module 102, the intermediate devices may be configured into any suitable arrangement.

The control module 102 can include, for example, a connector housing 112 and a sealed electronics housing 114. An electronic subassembly 110 and an optional power source 120 are disposed in the electronics housing 114. A control module connector 144 is disposed in the connector housing 112. The control module connector 144 is configured and arranged to make an electrical connection between the lead 103 and the electronic subassembly 110 of the control module 102.

In some embodiments, the control module 102 also includes one or more light sources 111 disposed within the sealed electronics housing 114. In alternate embodiments, the one or more light sources 111 are external to the control module such as, for example, disposed along the lead or in a unit attached to the lead. The one or more light sources can be, for example, a light-emitting diode (“LED”), laser diode, organic light-emitting diode (“OLED”), or the like. When the control module 102 includes multiple light sources, the light sources can provide light in at a same wavelength or wavelength band or some, or all, of the light sources can provide light at different wavelength or different wavelength bands. When the one or more light sources 111 are external to the lead(s) or disposed along the lead proximal to the light emitters 135, the light emitted by the light sources can be directed to one or more optical fibers or other light-transmitting body. The optical fiber, or a series of optical fibers, can transmit the light from the one or more light sources 111 through the control module 102 and lead 103 to the light emitter 135 (which can be terminus of the optical fiber). In at least some embodiments, the optical fiber is a single mode optical fiber. In other embodiments, the optical fiber is a multi-mode optical fiber. In some embodiments, the system includes a single optical fiber. In other embodiments, the system may employ multiple optical fibers in series or in parallel.

In other embodiments, the light emitter 135 can also be the light source (a light-emitting diode (“LED”), laser diode, organic light-emitting diode (“OLED”), or the like), or a combination of light sources, with conductors extending along the lead 103 and coupled to the electronic subassembly 110 to provide signals and power for operating the light source. In yet other embodiments, the light source can be disposed elsewhere in the control module 102, on the lead 103, in another element such as a lead extension, splitter, adaptor, or other stand-alone element.

The stimulation system or components of the stimulation system, including the lead 103 and the control module 102, can be implanted into the body of a patient. In other embodiments, the control module 102 may be external and in transcutaneous communication with the lead 103. The stimulation system can be used for a variety of applications including, but not limited to peripheral nerve stimulation, brain stimulation, deep brain stimulation, neural stimulation, spinal cord stimulation, muscle stimulation, sacral nerve stimulation, dorsal root ganglion stimulation, trigeminal nerve stimulation, occipital nerve stimulation, vagus nerve stimulation, pudendal nerve stimulation, sphenopalatine ganglion stimulation, sympathetic chain modulation, adrenal gland modulation, tibial nerve stimulation, splanchnic nerve stimulation, splenic nerve stimulation, peripheral field stimulation, other peripheral organ stimulation, and the like.

The one or more lead bodies 106 are made of a non-conductive, biocompatible material such as, for example, silicone, polyurethane, polyether ether ketone (“PEEK”), epoxy, and the like or combinations thereof. The one or more lead bodies 106 may be formed in the desired shape by any process including, for example, molding (including injection molding), casting, and the like.

One or more terminals (e.g., 310 in FIGS. 2A-2B) are typically disposed along the proximal end of the one or more lead bodies 106 of the stimulation system 100 (as well as any splitters, lead extensions, adaptors, or the like) for electrical connection to corresponding connector contacts (e.g., 314 in FIGS. 2A-2B). The connector contacts are disposed in connectors (e.g., 144 in FIGS. 1-2B; and 322 FIG. 2B) which, in turn, are disposed on, for example, the control module 102 (or a lead extension, a splitter, an adaptor, or the like). Electrically conductive wires, cables, or the like (not shown) extend from the terminals to the light emitter 135 or electrodes 134.

The electrically-conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the one or more lead bodies 106, for example, for inserting a stylet to facilitate placement of the one or more lead bodies 106 within a body of a patient. Additionally, there may be one or more lumens (not shown) that open at, or near, the distal end of the one or more lead bodies 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably sealable at the distal end.

FIG. 2A is a schematic side view of one embodiment of a proximal portion of one or more elongated devices 301 configured and arranged for coupling to one embodiment of the control module connector 144. The one or more elongated devices may include, for example, one or more of the lead bodies 106 of FIG. 1, one or more intermediate devices (e.g., a splitter, the lead extension 324 of FIG. 2B, an adaptor, or the like or combinations thereof), or a combination thereof.

The control module connector 144 defines at least one port into which a proximal end of the elongated device 301 can be inserted, as shown by directional arrows 312a and 312b. In FIG. 2A (and in other figures), the connector housing 112 is shown having two ports 304a and 304b. The connector housing 112 can define any suitable number of ports including, for example, one, two, three, four, five, six, seven, eight, or more ports.

The control module connector 144 also includes a plurality of connector contacts, such as connector contact 314, disposed within each port 304a and 304b. When the elongated device 301 is inserted into the ports 304a and 304b, the connector contacts 314 can be aligned with a plurality of terminals 310 disposed along the proximal end(s) of the elongated device(s) 301 to electrically couple the control module 102 to the electrodes (134 of FIG. 1) disposed on the paddle body 104 of the lead 103. Each of the terminals 310 can couple to the light emitter 135 or one or more of the electrodes 134. Examples of connectors in control modules are found in, for example, U.S. Pat. Nos. 7,244,150 and 8,224,450, which are incorporated by reference.

FIG. 2B is a schematic side view of another embodiment of the stimulation system 100. The stimulation system 100 includes a lead extension 324 that is configured and arranged to couple one or more elongated devices 301 (e.g., one of the lead bodies 106 of FIG. 1, a splitter, an adaptor, another lead extension, or the like or combinations thereof) to the control module 102. In FIG. 2B, the lead extension 324 is shown coupled to a single port 304 defined in the control module connector 144. Additionally, the lead extension 324 is shown configured and arranged to couple to a single elongated device 301. In alternate embodiments, the lead extension 324 is configured and arranged to couple to multiple ports 304 defined in the control module connector 144 (e.g., the ports 304a and 304b of FIG. 1), or to receive multiple elongated devices 301 (e.g., both of the lead bodies 106 of FIG. 1), or both.

A lead extension connector 322 is disposed on the lead extension 324. In FIG. 2B, the lead extension connector 322 is shown disposed at a distal portion 326 of the lead extension 324. The lead extension connector 322 includes a connector housing 328. The connector housing 328 defines at least one port 330 into which terminals 310 of the elongated device 301 can be inserted, as shown by directional arrow 338. Each of the terminals 310 can couple to the light emitter 135 or one or more of the electrodes 134. The connector housing 328 also includes a plurality of connector contacts, such as connector contact 340. When the elongated device 301 is inserted into the port 330, the connector contacts 340 disposed in the connector housing 328 can be aligned with the terminals 310 of the elongated device 301 to electrically couple the lead extension 324 to the electrodes (134 of FIG. 1) disposed along the lead (103 in FIG. 1).

In at least some embodiments, the proximal end of the lead extension 324 is similarly configured and arranged as a proximal end of the lead 103 (or other elongated device 301). The lead extension 324 may include a plurality of electrically-conductive wires (not shown) that electrically couple the connector contacts 340 to a proximal portion 348 of the lead extension 324 that is opposite to the distal portion 326. In at least some embodiments, the conductive wires disposed in the lead extension 324 can be electrically coupled to a plurality of terminals (not shown) disposed along the proximal portion 348 of the lead extension 324. In at least some embodiments, the proximal portion 348 of the lead extension 324 is configured and arranged for insertion into a connector disposed in another lead extension (or another intermediate device). In other embodiments (and as shown in FIG. 2B), the proximal portion 348 of the lead extension 324 is configured and arranged for insertion into the control module connector 144.

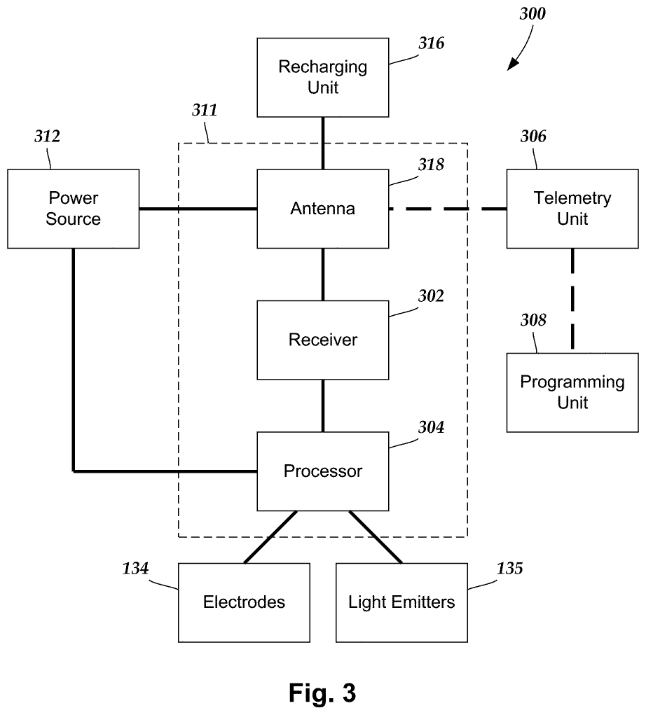

FIG. 3 is a schematic overview of one embodiment of components of an electrical/optical stimulation system 300 including an electronic subassembly 311 disposed within a control module. It will be understood that the electrical/optical stimulation system can include more, fewer, or different components and can have a variety of different configurations including those configurations disclosed in the stimulator references cited herein.

Some of the components (for example, a power source 312, an antenna 318, a receiver 302, and a processor 304) of the electrical/optical stimulation system can be positioned on one or more circuit boards or similar carriers within a sealed housing of an implantable pulse generator, if desired. Any power source 312 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 318 or a secondary antenna. The external power source can be in a device that is mounted on the skin of the user or in a unit that is provided near the user on a permanent or periodic basis.

If the power source 312 is a rechargeable battery, the battery may be recharged using the optional antenna 318, if desired. Power can be provided to the battery for recharging by inductively coupling the battery through the antenna to a recharging unit 316 external to the user. Examples of such arrangements can be found in the references identified above.

In one embodiment, light is emitted by the light emitter 135 of the lead body to stimulate nerve fibers, muscle fibers, or other body tissues near the electrical/optical stimulation system. The processor 304 is generally included to control the timing and other characteristics of the electrical/optical stimulation system. For example, the processor 304 can, if desired, control one or more of the intensity, wavelength, amplitude, pulse width, pulse frequency, cycling (e.g., for repeating intervals of time, determining how long to stimulate and how long to not stimulate), and electrode stimulation configuration (e.g., determining electrode polarity and fractionalization) of the optical stimulation.

Additionally, the processor 304 can select which, if not all, of the sensing electrodes are activated. Moreover, the processor 394 can control which types of signals the sensing electrodes detect. In at least some embodiments, the sensing electrodes detect a level of neuronal activation, or neuronal firing rates, or both, received directly from the target stimulation location. In other embodiments, the sensing electrodes detect one or more other signals received from the target stimulation location in addition to, or in lieu of the level of neuronal activation or neuronal firing rates, such as evoked compound action potentials, local field potentials, multiunit activity, electroencephalograms, electrophysiology, or electroneurograms. In at least some embodiments, one or more of the received signals (e.g., evoked compound action potentials, local field potentials, multiunit activity, electroencephalograms, electrophysiology, electroneurograms, or the like) can be used to indirectly measure the level of neuronal activation, or neuronal firing rates, or both, at the target stimulation location.

Optionally, the processor 304 can select one or more stimulation electrodes to provide electrical stimulation, if desired. In some embodiments, the processor 304 selects which of the optional stimulation electrode(s) are cathodes and which electrode(s) are anodes.

Any processor can be used and can be as simple as an electronic device that, for example, produces optical stimulation at a regular interval or the processor can be capable of receiving and interpreting instructions from an external programming unit 308 that, for example, allows modification of stimulation characteristics. In the illustrated embodiment, the processor 304 is coupled to a receiver 302 which, in turn, is coupled to the optional antenna 318. This allows the processor 304 to receive instructions from an external source to, for example, direct the stimulation characteristics and the selection of electrodes, if desired. Associated with the processor 304 is the stimulation components that generate the electrical and optical stimulation that is directed to the electrodes 134 and light emitter 135.

In one embodiment, the antenna 318 is capable of receiving signals (e.g., RF signals) from an external telemetry unit 306 which is programmed by the programming unit 308. The programming unit 308 can be external to, or part of, the telemetry unit 306. The telemetry unit 306 can be a device that is worn on the skin of the user or can be carried by the user and can have a form similar to a pager, cellular phone, or remote control, if desired. As another alternative, the telemetry unit 306 may not be worn or carried by the user but may only be available at a home station or at a clinician's office. The programming unit 308 can be any unit that can provide information to the telemetry unit 306 for transmission to the electrical/optical stimulation system 300. The programming unit 308 can be part of the telemetry unit 306 or can provide signals or information to the telemetry unit 306 via a wireless or wired connection. One example of a suitable programming unit is a computer operated by the user or clinician to send signals to the telemetry unit 306.

The signals sent to the processor 304 via the antenna 318 and the receiver 302 can be used to modify or otherwise direct the operation of the electrical/optical stimulation system. For example, the signals may be used to modify the stimulation characteristics of the electrical/optical stimulation system such as modifying one or more of stimulation duration, pulse frequency, waveform, and stimulation amplitude. The signals may also direct the electrical/optical stimulation system 300 to cease operation, to start operation, to start charging the battery, or to stop charging the battery. In other embodiments, the stimulation system does not include the antenna 318 or receiver 302 and the processor 304 operates as programmed.

Optionally, the electrical/optical stimulation system 300 may include a transmitter (not shown) coupled to the processor 304 and the antenna 318 for transmitting signals back to the telemetry unit 306 or another unit capable of receiving the signals. For example, the electrical/optical stimulation system 300 may transmit signals indicating whether the electrical/optical stimulation system 300 is operating properly or not or indicating when the battery needs to be charged or the level of charge remaining in the battery. The processor 304 may also be capable of transmitting information about the stimulation characteristics so that a user or clinician can determine or verify the characteristics.

The leads and control modules illustrated in FIGS. 1 to 2B and the system illustrated in FIG. 3 are arranged to provide both electrical and optical stimulation. It will be understood that these leads, control modules, and systems can be modified to provide only electrical stimulation or only optical stimulation by omitting components that are used solely for electrical or optical stimulation, respectively.

In at least some embodiments, the combination of electrical and optical stimulation can provide greater selectivity of the nerve fibers or other tissue that is activated or inhibited. For example, optical energy can be used to increase membrane potentials to increase the selectivity of electrical stimulation by illuminating the tissue to be stimulated optically during or prior to electrical stimulation.

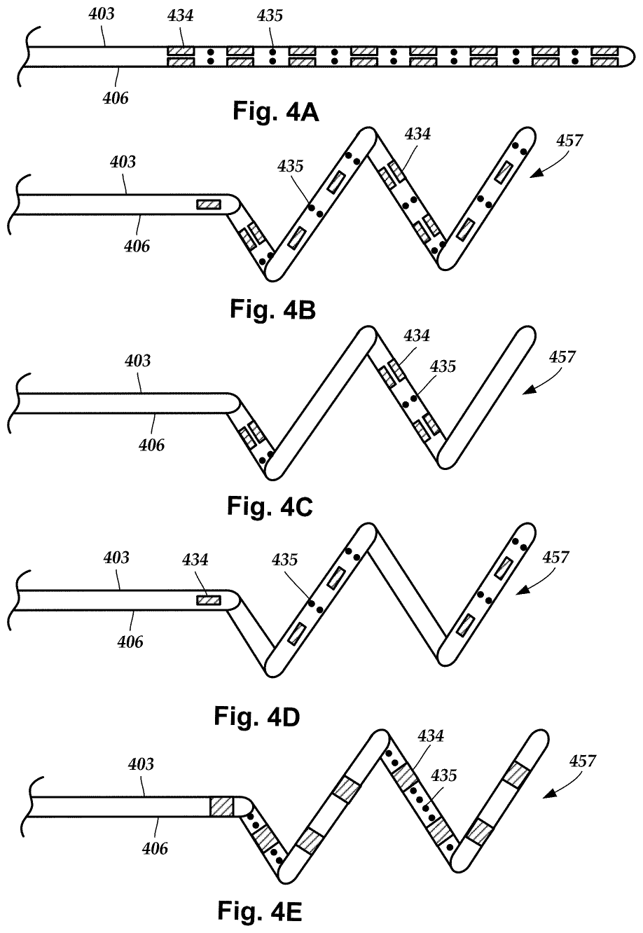

FIG. 4A illustrates the distal portion of one embodiment of a lead 403 with a lead body 406, electrodes 434, and light emitters 435. In FIG. 4A, the lead 403 is straight. The lead 403 can be implanted near or adjacent the nerve or tissue to be stimulated.

FIG. 4B illustrates the distal portion of another embodiment of a lead 403 which is not straight along the distal end portion of the lead, but instead forms a coil or spiral 457 along the distal end of the lead. In at least some embodiments, the lead 403 of Figure B may be implanted in the coiled or spiral configuration. In at least some other embodiments, the lead 403 is straightened during implantation. For example, in some embodiments, the lead 403 can be straightened by insertion of a straight stylet (not shown) within the lead. In some embodiments, the lead 403 can be straightened by insertion of the lead into a needle or cannula (not shown) that has an inner diameter that is smaller than the outer diameter of the coil or spiral 457 of the lead 403. (In some embodiments, the inner diameter of needle or cannula may be the same or slightly larger than the outer diameter of the lead body 406 of the lead 403.)

The lead 403, in the straightened configuration (see, e.g., lead 403 in FIG. 4A) can be implanted through a lead introducer, such as a needle or cannula, using a relatively non-invasive implantation method. When the stylet, cannula, or needle is removed after implantation, the lead 403 assumes the coiled or spiral configuration of FIG. 4B. The lead 403 can be made of a spring-like or shape memory material, or can include a coil or shape memory material in the lead body 403, so that the distal portion of the lead body 403 assumes the coil or spiral shape illustrated in FIG. 4B absent the stylet, needle, or cannula straightening the lead.

In at least some embodiments, the lead 403 can be implanted so that when the stylet, needle, or cannula is removed, the distal portion of the lead coils or spirals around, or next to, a nerve or other tissue that is to be stimulated. In at least some embodiments, the lead 403 may be implanted adjacent a peripheral nerve or within a body sheath, such as the carotid sheath, or other body cavity that allows the distal portion of the lead 403 to form the coil or spiral 457 and be adjacent to, or in contact with, the tissue to be stimulated. For example, a lead implanted in the carotid sheath can be positioned so that it is adjacent to, or coiled or spiraled around, or otherwise in contact with, the vagus nerve that extends within the carotid sheath.

In the leads 403 of FIGS. 4A and 4B, the electrodes 434 and light emitters 435 are disposed around the entire circumference of the lead. In contrast, in FIG. 4C, the lead 403 has electrodes 434 and light emitters 435 disposed only, or primarily, on the portion of the lead that forms an interior of the coil or spiral 457. Such an arrangement can be particularly useful when, for example, the lead 403 is arranged to coil or spiral around a nerve. In FIG. 4D, the lead 403 has electrodes 434 and light emitters 435 disposed only, or primarily, on the portion of the lead that forms an exterior of the coil or spiral 457. Such an arrangement can be particularly useful when, for example, the lead 403 is arranged to be disposed adjacent a nerve or other tissue to be stimulated.

In FIGS. 4A to 4D, the electrodes 434 are segmented electrodes and the light emitters 435 form a set of rings around the circumference of the lead. In FIG. 4E, the electrodes 434 are ring electrodes and the light emitters 435 are arranged longitudinally along the length of the lead. It will be recognized that a lead can include any combination of segmented electrodes and ring electrodes. It will also be recognized that the light emitters can be arranged circumferentially (FIGS. 4A to 4D), longitudinally (FIG. 4E), or any combination thereof or in any other arrangement (for example, in a spiral arrangement around the circumference of the lead).

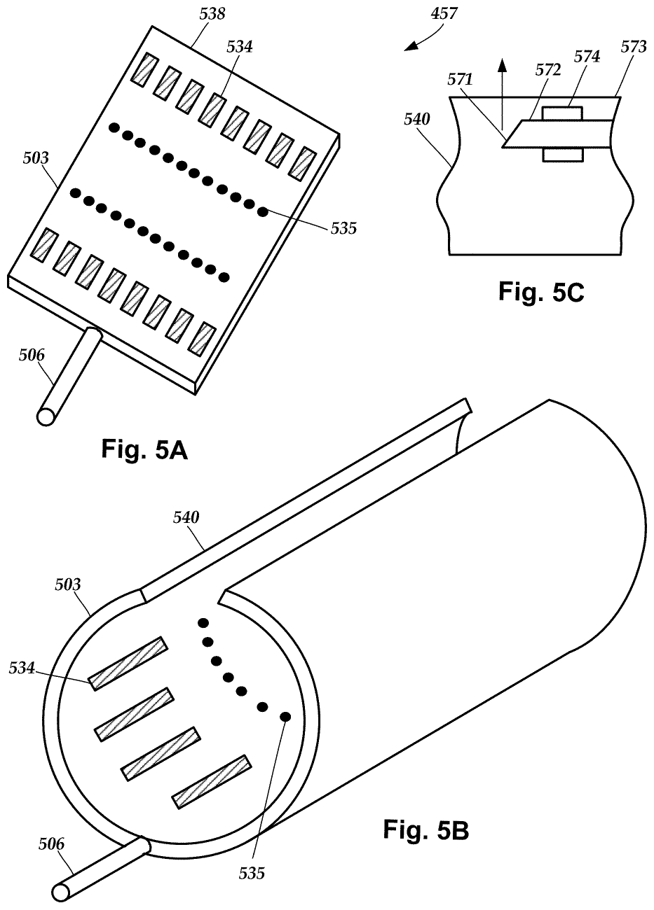

FIGS. 5A and 5B illustrate a distal end of another lead 503 that includes a lead body 506 and can be a paddle lead with a paddle 538 (FIG. 5A) or a cuff lead with a cuff 540 (FIG. 5B). The lead 503 includes one or more rows of electrodes 534 and one or more rows of light emitters 535 disposed on a surface 542 of the paddle 538 or cuff 540. It will be recognized that instead of rows extending across the width of the paddle 538 or cuff 540, the electrodes 534 or light emitters 535 can be arranged in columns extending along the length of the paddle or cuff or in any other suitable arrangement of electrodes or light emitters on the paddle or cuff. In FIG. 5B, the electrodes 534 and light emitters 535 are arranged on an interior surface 573 of the cuff 540 that fits around a nerve.

In at least some embodiments that utilize optical fibers as light emitters in a cuff 540 (or a paddle 538), the distal end 571 of the optical fiber 572 may be cut at an angle to direct light toward the nerve or tissue as illustrated in FIG. 5C. In at least some embodiments, a mechanical anchor 574 may be attached near the distal end of the optical fiber and embedded in the body of the paddle or cuff to resist rotation or displacement of the optical fiber.

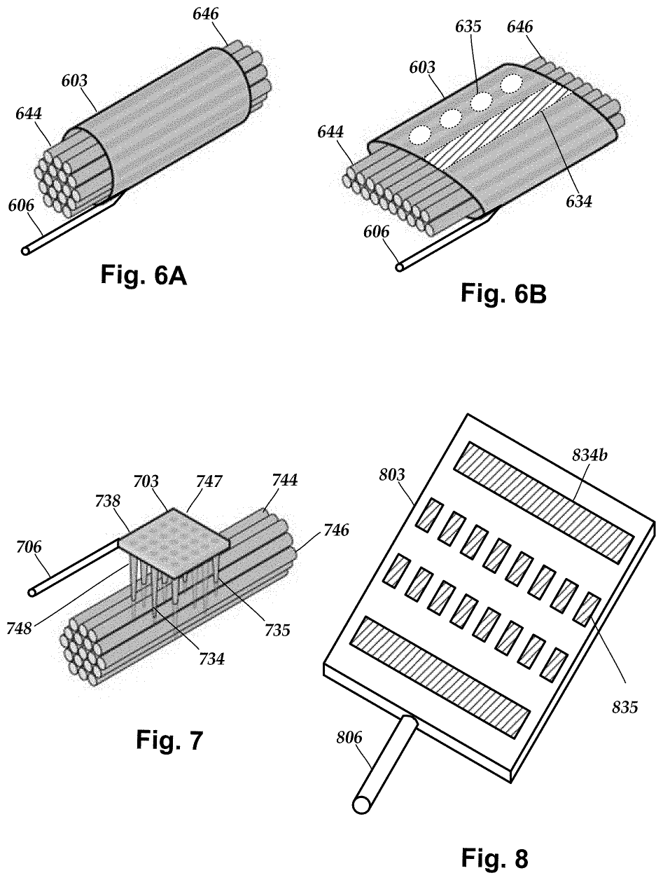

FIGS. 6A and 6B illustrate another example of a distal end of a cuff lead 603. FIG. 6A illustrates the cuff lead 603 as it is initially placed around a nerve 644. After placement, the cuff lead 603 puts pressure on the nerve fibers 646 of the nerve 644 to flatten the nerve so that more nerve fibers are in contact with the cuff lead 603, as illustrated in FIG. 6B. The cuff lead 603 includes a lead body 606 as well as one or more electrodes 634 and one or more light emitters 635 disposed on the interior of the cuff lead. In the illustrated embodiment of FIG. 6B, the electrode 634 and light emitters 635 extend in columns along a length of the interior of the cuff lead, but it will be understood that other embodiments can include the electrodes and light emitters extending in rows along a width of the cuff lead (such as illustrated in FIG. 5B) or in any other suitable arrangement.

FIG. 7 illustrates another embodiment of a distal end of a lead 703 that includes a lead body 706 and a paddle 738 (or cuff) that includes a body 747 with one or more needle-like extensions 748 extending from the body. In at least some embodiments, one or more of the needle-like extensions 748 may be metal or include a metal portion that can act as an electrode 734. In at least some embodiments, at least one of the light emitters (for example, a LED or end of an optical fiber) can be arranged to emit light from one or more the needle-like extensions 748. The needle-like extensions 748 can be inserted into a nerve 744 or between, or into, nerve fibers 746 for more direct delivery of the electrical or optical stimulation. In some embodiments, the lead 703 can be a cuff lead. In some embodiments, the lead 703 may be configured to flatten the nerve in a manner similar to that illustrated in FIG. 6B.

FIG. 8 illustrates an embodiment of a distal end of a lead 803 that includes a lead body 806, one or more segmented electrodes 834a, and one or more long electrodes 834b. The lead 803 can be a paddle lead (similar to paddle lead 503 of FIG. 5A) or can be wrapped around a nerve to form a cuff lead (similar to cuff lead 503 of FIG. 5B). In at least some embodiments, each of the long electrodes 834b can extend at least 50%, 60%, 70%, 80%, 85%, 90%, 95% or longer along either the width or length of the paddle or cuff. In at least some embodiments, the lead 803 can include one or more light emitters similar to those disclosed in lead 503 of FIGS. 5A and 5B.

In some embodiments, one or more of the segmented electrodes 834a can be selectively used to stimulate different nerve fibers. In contrast, particularly when the lead is a cuff lead, either of the long electrodes 834b can be used to block action potentials from traveling down the nerve in one direction. Thus, the combination of segmented electrodes 834a and long electrodes 834b can provide for the selection stimulation of nerve fibers while concurrently blocking action potentials in one direction. As an example, the lead 803 implanted as a cuff lead around the vagus nerve can selectively stimulation afferent nerve fibers while blocking actions potentials directed toward the visceral organs.

The above specification and examples provide a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. A stimulation lead, comprising:
a lead body having a distal portion and a proximal portion;
a plurality of light emitters disposed along the distal portion of the lead body and configured to emit light; and
a plurality of stimulation electrodes disposed along the distal portion of the lead body;
wherein a portion of the lead body upon which at least one of the light emitters and at least one of the electrodes is disposed is arranged to form a coil or spiral.

2. The stimulation lead of claim 1, wherein a plurality of the stimulation electrodes are segmented electrodes.

3. The stimulation lead of claim 2, wherein all of the segmented electrodes are positioned on a surface of the lead which forms an interior surface of the coil or spiral.

4. The stimulation lead of claim 2, wherein all of the segmented electrodes are positioned on a surface of the lead which forms an exterior surface of the coil or spiral.

5. A kit, comprising:
the stimulation lead of claim 1; and
a straightening stylet configured and arranged to straighten the coil or spiral of the stimulation lead when the straightening stylet is disposed in the stimulation lead.

6. A kit, comprising:
the stimulation lead of claim 1; and
a needle or cannula configured to receive the stimulation lead and to straighten the coil or spiral of the stimulation lead when the stimulation lead is received in the needle or cannula.

7. A system, comprising:
the stimulation lead of claim 1; and
a control module coupleable to the stimulation lead, the control module configured to direct the emission of the light from the light emitters of the lead and the delivery of electrical stimulation using the electrodes of the lead.

8. A stimulation lead comprising:
a lead body;
a paddle or cuff attached to the lead body;
a plurality of light emitters disposed on the paddle or cuff and configured to emit light; and
a plurality of stimulation electrodes disposed on the paddle or cuff.

9. The stimulation lead of claim 8, wherein the light emitters are arranged in at least one row or column on the paddle or cuff and the stimulation electrodes are arranged in at least one row or column on the paddle or cuff.

10. The stimulation lead of claim 8, wherein the light emitters are optical fibers, wherein each optical fiber is angled at a distal end of the optical fiber to emit light in a selected direction.

11. The stimulation lead of claim 10, further comprising at least one mechanical anchor coupled to each of the optical fibers and disposed in the paddle or cuff to hinder rotation of the optical fiber within the paddle or cuff.

12. The stimulation lead of claim 8, wherein the paddle or cuff is the cuff and the cuff is configured to apply pressure on a portion of a nerve disposed within the cuff to flatten the portion of the nerve.

13. The stimulation lead of claim 8, wherein the paddle or cuff comprises a body and one or more needle-like extensions extending from the body, wherein at least one of the stimulation electrodes is disposed on at least one of the needle-like extensions.

14. The stimulation lead of claim 8, wherein the paddle or cuff comprises a body and one or more needle-like extensions extending from the body, wherein at least one of the light emitters is configured to emit light from at least one of the needle-like extensions.

15. A system, comprising:
the stimulation lead of claim 8; and
a control module coupleable to the stimulation lead, the control module configured to direct the emission of the light from the light emitters of the lead and the delivery of electrical stimulation using the electrodes of the lead.

16. A stimulation lead, comprising:
a lead body;
a paddle or cuff attached to the lead body;
at least one long electrode disposed on the paddle or cuff and extending at least 50% of a width or length of the paddle or cuff; and
a plurality of segmented stimulation electrodes disposed in at least one row or column on the paddle or cuff.

17. The stimulation lead of claim 16, wherein the at least one long electrode is two long electrodes and the segmented stimulation electrodes are disposed between the two long electrodes.

18. The stimulation lead of claim 16, wherein the paddle or cuff is the cuff and the at least one long electrode extends at least 80% of the width or length of the cuff.

19. The stimulation lead of claim 16, further comprising at least one light emitter disposed on the paddle or cuff.

20. A system, comprising:
the stimulation lead of claim 16; and
a control module coupleable to the stimulation lead, the control module configured to direct the delivery of electrical stimulation using the electrodes of the lead.
